(19) United States
(12) Patent Application Publication
(10) Pub. No.: US 2003/0163197 A1
(43) Pub. Date: Aug. 28, 2003

(54) Detachable multi-chamber breast form with permanently grown adhesive

(76) Inventor: David Chen, Walnut, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/085,917

(22) Filed: Feb. 28, 2002

(51) Int. Cl. A61F 2/52

(52) U.S. Cl. 623/7; 450/57

(57) ABSTRACT

An breast form having a plurality of independent and detachable/separable chambers, where each of the chambers is formed from a thermoplastic film material. A volume of deformable substance is sealably disposed within each of the chambers; and a pressure-sensitive adhesive is permanently grown to an interior surface of each of the chambers, such that the pressure-sensitive adhesive detachably adjoins to either an exterior surface of an adjacent chamber or a user's skin.

### FIELD OF THE INVENTION

The present invention relates to an improved breast form, particularly a detachable multi-chamber breast form with a permanently grown adhesive.

### BACKGROUND OF THE INVENTION

Women who, for whatever reason, are not satisfied with the size of their own breasts and desire larger, more shapely breasts must select among two alternative methods for enhancing their breast size, by either using rudimentary externally worn articles, such as foam pads and the like, or by undergoing a surgical operation to be fitted with a breast implant. Opting for use of a surgical breast implant carries with it the danger inherent in any surgical operation and can be quite expensive. In addition to the dangers inherent with the surgical operation is the potential health dangers that may be associated with using a particular type of breast implant, namely, the silicone breast implant. Accordingly, women wishing to enhance their physical appearance in a non-permanent and health-risk free manner opt to use one of the many types of externally worn articles.

A key feature of such externally worn article is that it look and feel natural so as to complement and not detract from the existing female breast that it is used to enhance. In addition to enhancing an existing breast, externally worn articles are designed to replace a female human breast that has been surgically removed. Externally worn articles that can be worn for the purpose of either enhancing or replacing human breasts are referred to a breast forms, and include a wide range of breast enhancers, breast inserts, and breast prostheses. A popular type of breast form has been made from a silicone gel material that is completely encased by plastic film material. The advantage of this type of breast form is that it looks like a natural human breast when worn and feels natural to the user, thus enhancing the self image and confidence of the user. Other breast forms, such as foam pads, water-filled pads and the like, do not afford the user these important qualities but, rather, look unnatural and feel foreign.

An additional limitation of known silicone gel breast forms has been the ability to make the breast forms lighter in weight, yet still maintain a desirable appearance. For example, it is common to experience severe discoloration in the breast form if silicone gel material is replaced with a lighter, A “filler” material. Because merely covering the breast forms with a fabric or material has not been desirable, there remains a need for a breast form that can be made from a lightweight material, yet still maintain a desirable appearance and function.

Known efforts to solve these problems have included providing a breast form that has multiple chambers that are permanently joined together. In such breast forms, there may be a different material in each chamber, thereby allowing the weight of the breast form to be reduced by having one chamber made from a lighter weight material. However, the known multiple chamber breast forms do not allow the user to customize the positioning of the chambers with respect to one another, nor interchange different chambers within the breast form depending on the shape or weight desired by the user. Accordingly, there exists a need for a multi-chamber breast form that allows the user to customize the breast form to their particular body shape by positioning the chambers independently, as well as customizing the look, feel, and weight of the breast form by choosing which chambers to include as part of the breast form.

### SUMMARY OF THE INVENTION

The present invention provides a breast form having a plurality of chambers, where each chamber encases a volume of substance between its interior surface and its exterior surface. Each chamber has a pressure-sensitive adhesive permanently grown to its interior surface that is designed to detachably adjoin to the exterior surface of an adjacent chamber or a user's skin. Because the chambers are separated, the user can position each chamber individually with respect to the other chamber(s) and the user's skin. Accordingly, the present breast form provides a nearly infinite number of possible configurations to accommodate the unique needs and preferences of each user.

The chambers are preferably made from a thermoplastic film material such as polyurethane. The volume of substance that is sealed within the chambers can be a wide variety of substances, including liquids, gels, and gases. Preferably, the chamber positioned farthest from the user will have a silicon gel material encased in it, and the chamber(s) positioned closer to the user can be made from a lighter substance, such as air, foam, or a silicon gel having a lightweight filler.

The ability to attach and detach the chambers from each other and the user's skin is achieved by the permanently grown pressure-sensitive adhesive. The pressure-sensitive adhesive can be made from a wide range of compositions suitable for such applications, but is preferably made from the family of fully cross linked organo-polysiloxanes. Further, the pressure-sensitive adhesive will have an adhesion force to the surface it is permanently grown to that is greater than a cohesion force to the surfaces to which it is detachably adjoined.

The user of the present breast form can create a breast form suitable for different circumstances. For example, the user may prefer a lighter breast form while exercising, or a more natural looking breast form while wearing a formal evening gown. Several other features are available with the present multi-chamber construction, such as providing void portions in the interior surface of one or more chambers, which will create an air bubble(s) between the chamber(s) having the void portions and the other chamber it is adjoined to, thereby reducing the weight of the breast form. These and other advantages over the known permanently attached multi-chamber breast forms will be apparent from the description below.

### BRIEF DESCRIPTION OF THE DRAWINGS

These and other features and advantages of the present invention will become appreciated as the same becomes better understood with reference to the drawings wherein:

FIG. 1 is side view of a breast form according to the present invention where the multiple chambers are detached;

FIGS. 2a-d show front views of an interior surface of a single chamber of the present breast form having a pressure-sensitive adhesive adjoined to the chamber in various formats;

FIGS. 3a-b show two chambers of the present breast form in detached and attached positions, respectively;

FIG. 4 shows a front chamber and a rear chamber positioned relative to each other;

FIGS. 5a-c show the front chamber and rear chamber of FIG. 4 positioned in different configurations;

FIGS. 6a-b show a front chamber and a smaller rear chamber in detached and attached positions, respectively;

FIGS. 7a-b show a front chamber and a larger rear chamber in detached and attached positions, respectively;

FIGS. 8a-b show a front chamber, a middle chamber, and a rear chamber in detached and attached positions, respectively;

FIG. 9 shows a single chamber of the present breast form;

FIGS. 10a-b show a front chamber and a rear chamber in detached and attached positions, respectively, where the front chamber has a void portion; and

FIGS. 11a-b show a front chamber and a rear chamber in detached and attached positions, respectively, where the front chamber has two void portions.

### DETAILED DESCRIPTION

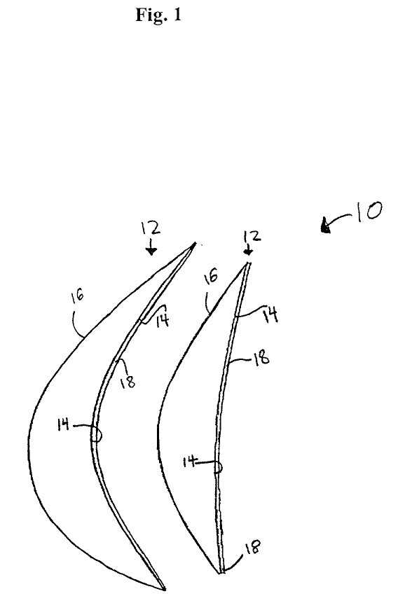

Multi-chamber breast forms, constructed according to the principles of this invention, generally comprise two or more detachable chambers each having a permanently grown layer of adhesive disposed thereon for purposes of attaching the chambers together in a variety of positions. As shown in FIG. 1, an example embodiment breast form 10 comprises two or more independent chambers 12. Each chamber 12 is entirely separated from the other chamber(s) 12, yet is also designed to cooperatively engage with adjacent chamber(s) in order to combine to make a single breast form 10. While the exact number of chambers 12 used to make the breast form 10 can vary depending on the desired configuration, in a preferred embodiment the breast form 10 will have two, three, or four chambers 12. Moreover, the breast form 10 is intended to serve as a full-range of externally worn articles that can be used to enhance or replace a user's breasts. Accordingly, breast forms of this invention can be configured as and/or used in the capacity of breast inserts, breast enhancers, and breast prostheses.

Each chamber 12 defines two surfaces relative to the user of the breast form, namely an interior surface 14 facing towards the user's skin, and an exterior surface 16 facing opposite the interior surface and away from the user's skin. The interior surface 14 of each chamber 12 comprises an adhesive layer 18. The adhesive layer 18 is a pressure-sensitive adhesive (PSA) that is permanently grown directly to the interior surface 14 of each chamber. The adhesive layer can be made from any type of PSA, but is preferably from the family of fully cross linked organo-polysiloxanes. Additionally, the adhesive layer 18 is completely re-usable and detachable, allowing the adhesive layer to be repeatedly attached and detached from the user's skin and from the exterior surface 16 of other chambers, which (as discussed below) can also include an adhesive layer. Accordingly, the adhesive layer 18 has an adhesion force to the interior surface 14 that is greater than a cohesion force to either the user's skin or the exterior surface 16 of an adjacent chamber. Further, the adhesive layer is able to withstand tremendous movement and pressure from the user without slipping and can even be subjected to water or sweat without degeneration of the adhesive properties. In fact, if the adhesive layer 18 becomes dirty (e.g., collects unwanted particles such as dust, lint, or debris), it can be cleaned with soap and water to remove the unwanted particles and fully restore the adhesive properties.

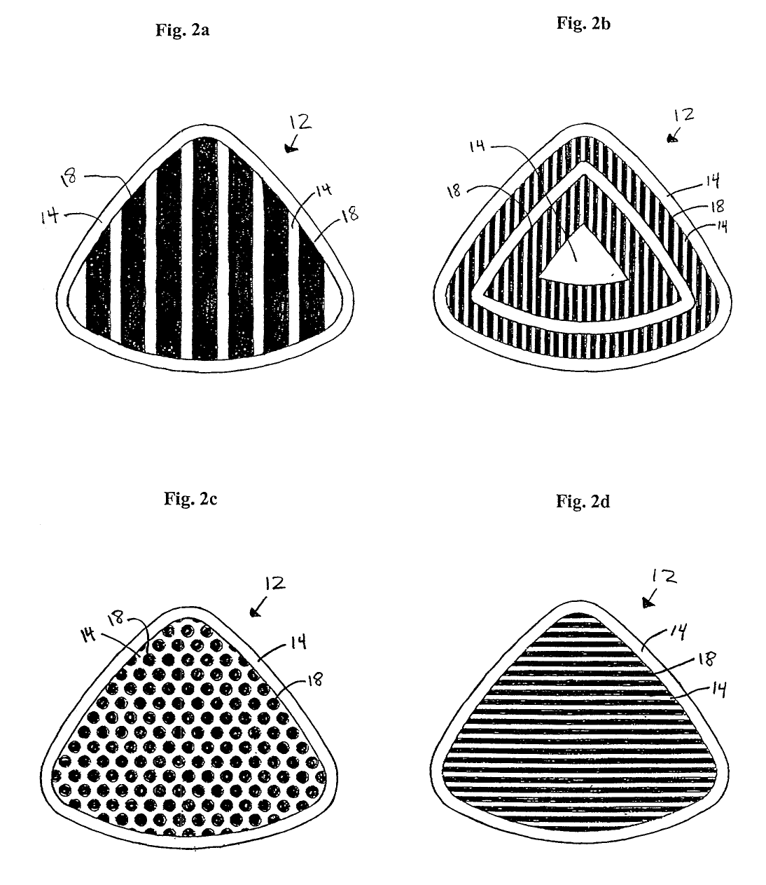

The thickness of the adhesive layer 18 that is grown to the interior surface can vary, as can the portions of the interior surface having the adhesive layer. Various factors can contribute to the thickness and placement of the adhesive layers such as the size, shape, and weight of each chamber. Preferably, the adhesive layer 18 of each chamber has a thickness of 1 to 200 millimeters. Further, in an example embodiment, the adhesive layer can cover more than 50% of the interior surface of each chamber, and can preferably have the adhesive layer positioned in portions substantially throughout of the entire interior surface of each chamber to allow for maximum adjustability of each chamber relative to one another and the user's skin. However, no particular configuration or amount of the adhesive layer on the interior surface is required. For example, referring to FIGS. 2a-d, front views of the interior surface 14 of the chamber 12 is shown, wherein the adhesive layer 18 is grown to the interior surface in vertical stripes (FIG. 2a), in vertical stripes with geometric designs (FIG. 2b), in a series of dots (FIG. 2c), and in horizontal stipes (FIG. 2d). It is also understood that the adhesive layer can be grown in a single large patch and cover the entire interior surface of the chamber. Because the breast form 10 comprises multiple, detachable chambers 12, the breast form can provide an infinite combination of breast sizes and shapes to accommodate the particular needs of each user. This customization is particularly useful when constructing the breast form for women who have undergone a mastectomy because post-mastectomy patients rarely have identical breast configurations after breast portions have been surgically removed. Although the breast form 10 will be described below as having two or three chambers 12, it is to be understood that multi-chamber breast forms of this invention are not limited to the illustrative configurations because the breast form is intended to accommodate the unique needs of each user.

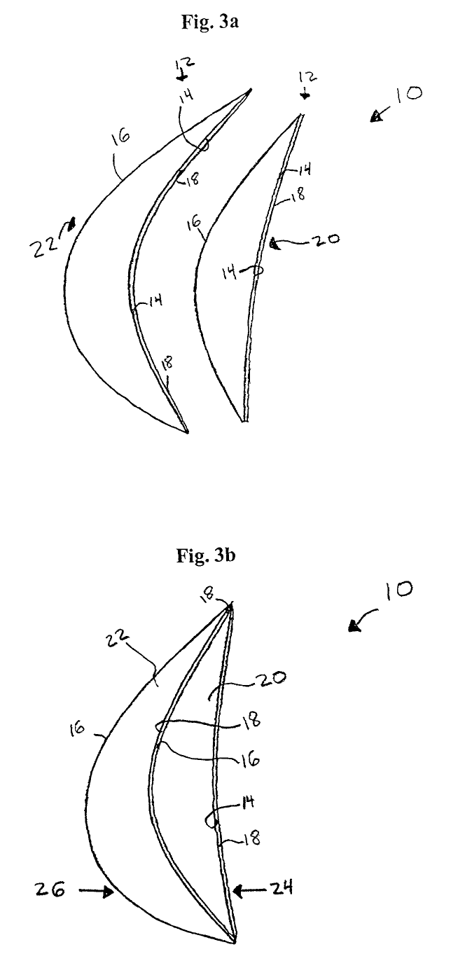

Referring to FIG. 3a, two chambers 12 are shown in detached form. FIG. 3b shows the two chambers of FIG. 3a combined as breast form 10. Just as each chamber 12 defines two surfaces relative to the user (i.e. the interior surface and the exterior surface), the individual chambers also define separate positions relative to the user. Specifically, the chamber nearest the user defines a rear position 20, and the chamber farthest from the user defines a front position 22. The chamber in the rear position 20 will be referred to as the rear chamber 20, and the chamber in the front position 22 will be referred to as the front chamber 22. The rear chamber 20 will have its interior surface 14 adjacent the user's skin and its exterior surface 16 adjacent the interior surface of the front chamber 22. Thus, the adhesive layer 18 of the front chamber will adjoin to the exterior surface of the rear chamber.

The combined chambers 12 shown in FIG. 3b define the breast form 10. Similar to each individual chamber, the breast form 10 defines two surfaces relative to the user, namely a rear surface 24 facing towards the user's skin, and a front surface 26 facing opposite the rear surface and away from the user's skin. The position and orientation of the chambers relative to each other will determine the shape and orientation of the breast form. Moreover, the shape and size of each chamber will determine how the user combines the chambers. So long as the interior surface of the front chamber has the adhesive layer permanently grown thereto, the front chamber can be adjoined to the rear chamber at any point along the exterior surface of the rear chamber. It may also be desirable to have the adhesive layer 18 grown to a portion of the exterior surface 16 of one of the chambers of the breast form, other than the front chamber 22, in order to provide additional adhesion between adjacent chambers. Similar to the adhesive layer 18 adjoined to the interior surface 14, the adhesive layer can be adjoined to the exterior surface 16 in a wide range of configurations, thicknesses, and amounts. The adhesive layer 18 of one chamber can be adjoined and removed from the adhesive layer of an adjacent chamber without damaging either adhesive layer because the adhesive layer 18 has a greater adhesion force to the surface it is permanently grown to than it has a cohesion force to any other surface.

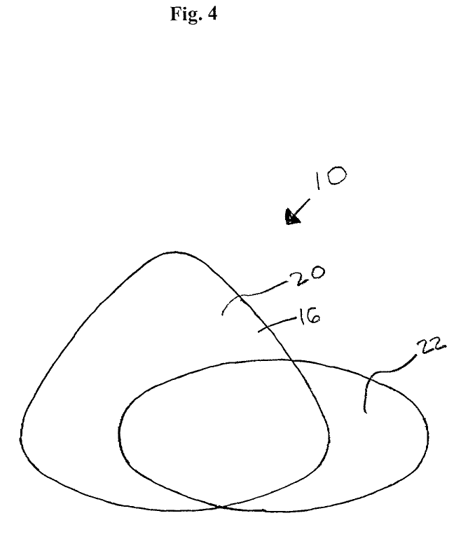

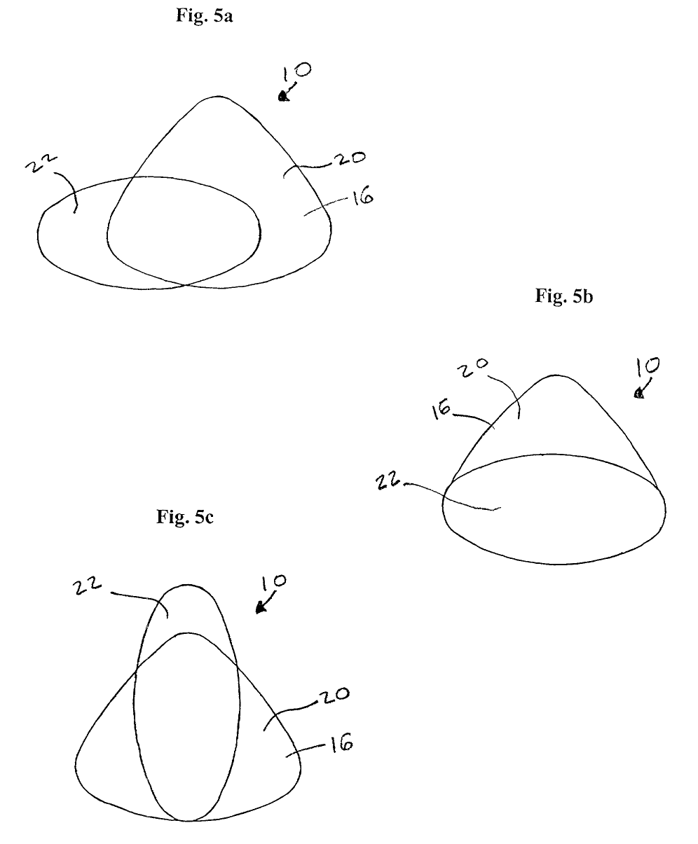

The entire interior surface of the front chamber 22 is shown adjoined to the exterior surface of the rear chamber 20. However, as shown in FIG. 4, less than the entire interior surface of the front chamber 22 can be adjoined to less than the entire exterior surface of the rear chamber 20. In such a configuration, the portion of the interior surface of the front chamber 22 that is not adjoined to the rear chamber 20 will be adjoined to the user's skin. It is understood that where the front chamber 22 overlaps with the rear chamber 20, the interior surface of the front chamber will be adjoined to the exterior surface of the rear chamber. Other sample configurations of how the front chamber and rear chamber can be arranged relative to each other are shown in FIGS. 5a-c.

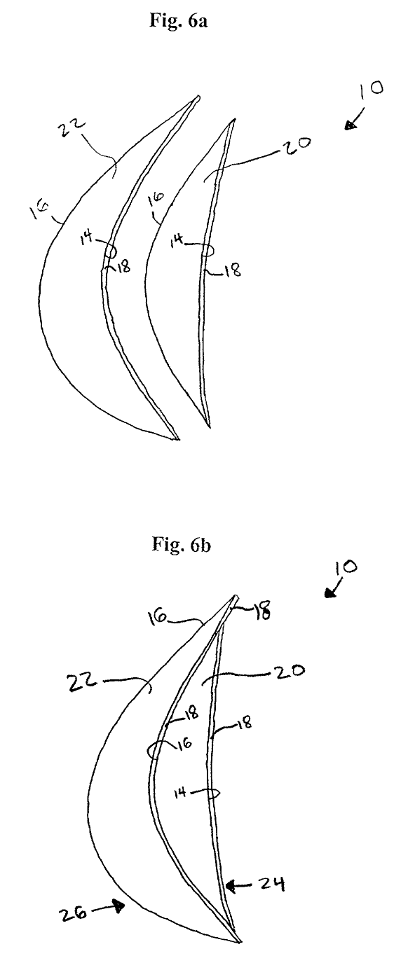

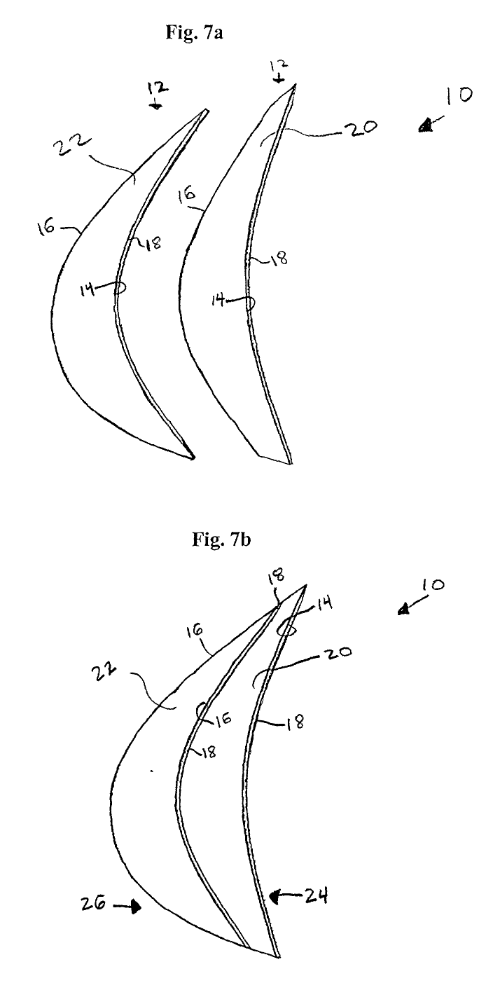

In addition to the various possible arrangements of the chambers 12 relative to each other, the sizes and shapes of the chambers can also be varied. As shown in FIG. 3b, the rear chambers and the front chamber are designed to fully match one another when combined as breast form 10. In FIGS. 6a-b, the rear chamber 20 is smaller than the front chamber 22, and FIGS. 7a-b show the rear chamber 20 larger than the front chamber 22. It is understood that the two chambers are adjoined in the same manner as described above, and can be adjoined in different orientations and have entirely different shapes and sizes.

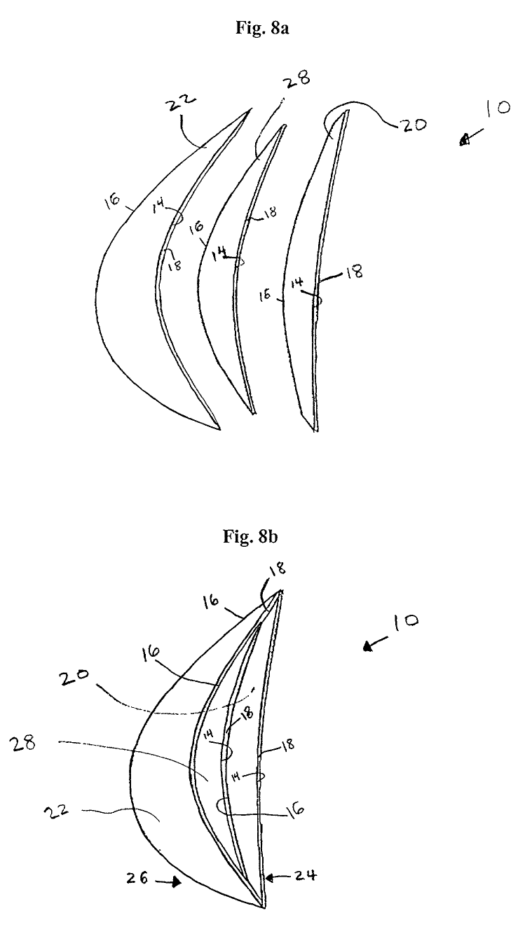

It should be apparent that there are no limitations on the sizes and shapes of the chambers 12, nor how the chambers 12 can be combined together. Because the chambers can be offset in any direction with no predetermined formats, there are infinite combinations, which is ideal for creating a breast form that is unique to each user. While the breast form 10 has been described as having two chambers 12, it is possible to have as many chambers as desired. An example of the breast form having three separate detachable chambers 12 is shown in FIGS. 8a-b. In this configuration, the third chamber defines an additional position relative to the user, namely a middle position 28, and the chamber in the middle position will be referred to as the middle chamber 28. The exterior surface of the middle chamber 28 is adjoined to the interior surface of the front chamber 22, and the interior surface of the middle chamber is adjoined to the exterior surface of the rear chamber 20. Just as described above with respect to two separate detachable chambers, each of the three chambers can be offset in any direction to provide infinite combinations. The three chamber breast form 10 is shown with all three chambers aligned with one another, and the middle chamber 28 is smaller and designed to fit between the front chamber and rear chamber. However, regardless of the number of chambers in the breast form 10, each chamber can be designed with a different size and shape.

The present breast form 10 also contemplates the possibility of each chamber 12 being made from a different material, or having some or all of the chambers being made from the same material. A key feature is the that the breast form 10 provides flexibility in design and customization by the user.

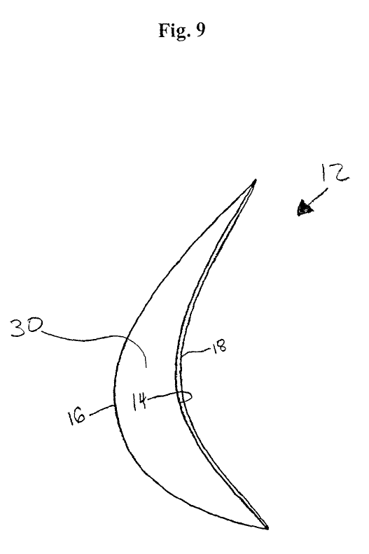

The general construction of each chamber 12 of the breast form 10 will be described with reference to the single chamber 12 shown in FIG. 9. It is understood, however, that all of the features discussed for chamber 12 are fully applicable to all embodiments of the breast form. Preferably, the chamber 12 is made of a flexible thermoplastic film material, such as polyurethane, and encases a volume of a deformable substance 30. The interior surface 14 and the exterior surface 16 can be formed from a single sheet of thermoplastic material that is heat sealed around overlapping edges of the single sheet, or can be made from two or more sheets of the thermoplastic film material.

The preferred method of making the present chamber 12 is the patented method disclosed in U.S. Pat. No. 5,693,164, entitled “Method of Making Breast Enhancers.” The teachings and disclosures of U.S. Pat. No. 5,693,164 are fully incorporated herein by reference. It is understood, however, that the chamber 12 can be made by any other suitable method, and is not limited to being manufactured according to the method disclosed in U.S. Pat. No. 5,693,164, nor is the chamber limited to encasing the types of materials disclosed in the patent. For example, the present chamber 12 can be made from any suitable material for encasing a non-solid material, such as a wide range of polymers, or even combinations between polymers and fabrics that are suitable for breast forms, such as four-way stretchable fabrics. Additionally, it is understood that each of the chambers 12 in the breast form 10 can be made independently of the other.

With respect to the deformable substance 30 encased within the chamber 12, the preferred deformable substance 30 for any given chamber may include a foam, a liquid, a gel, a fabric, or a gas. It is preferred that at least one of the chambers of the breast form, preferably the front chamber 22, have a volume of a silicone gel material encased therein. The silicone gel material can comprise a wide range of silicone based compositions suitable for breast forms. As disclosed in U.S. Pat. No. 5,693,164, the silicone gel material inside of the breast form results from curing various compositions and amounts of silicone oils, resins, inhibitors, catalysts and pigments. The structure of the chamber of the present breast form is intended to be compatible with any type of silicone composition suitable for enhancing or replacing a user's breasts. For example, one type of suitable silicone composition is taken from the family of fully cross-linked organo-polysiloxanes.

In the situation where the front chamber 22 has silicone gel material encased within it, it may be desirable to have the chamber(s), such as the rear chamber 20 and the middle chamber 28, encasing a different deformable substance. For example, in the case of the breast form having two chambers, such as shown in FIG. 3b, the front chamber 22 can have the silicone gel material encased therein, while the rear chamber 20 can have a foam or a lightweight silicone gel material encased in the chamber. One suitable lightweight silicone gel material can comprise greater than about 40 percent by volume of a low-density filler, and less than about 60 percent by volume of organo-polysiloxane. A suitable filler can be one having a density of approximately 0.01 g/cm³. Using such a material in the rear chamber could allow the breast form to appear to be made completely from silicone gel material, yet actually have a considerably lighter weight because the rear chamber 20 is made form a lighter material. Additionally, the user could have different rear chambers 20 that are used for different occasions, such as having the rear chamber filled with silicone gel for more formal occasions, and filled with foam or a less attractive lightweight silicone material for more casual occasions. The user can choose the combination of chambers depending on what the user will be doing (i.e. sitting, walking, running, swimming, etc.) and what the user will be wearing (i.e. thick sweater, thin blouse, bathing suit, etc.).

In the situation of the breast form having three chambers, as shown in FIGS. 8a-b, it may be desirable to have the middle chamber 28 encasing a different deformable material 30 than the front chamber and the rear chamber. For example, the middle chamber could be filled with air, which would provide an extremely lightweight chamber, yet help provide additional size to the breast form.

Overall, each chamber of the breast form can be made from a wide range of materials to construct the interior and exterior surfaces, and can be filled with a wide range of substances or materials. Because the chambers 12 are all detachable and have more than 50% of their interior surface covered with the adhesive layer 18, users of the breast form 10 have tremendous flexibility in designing their breast forms and can change the appearance and feel of the breast forms as regularly as they desire simply by using a different chamber or repositioning the chambers. Unlike breast forms having multiple chamber permanently attached to one another, the present breast form allows complete customization of size, fit, feel, and appearance.

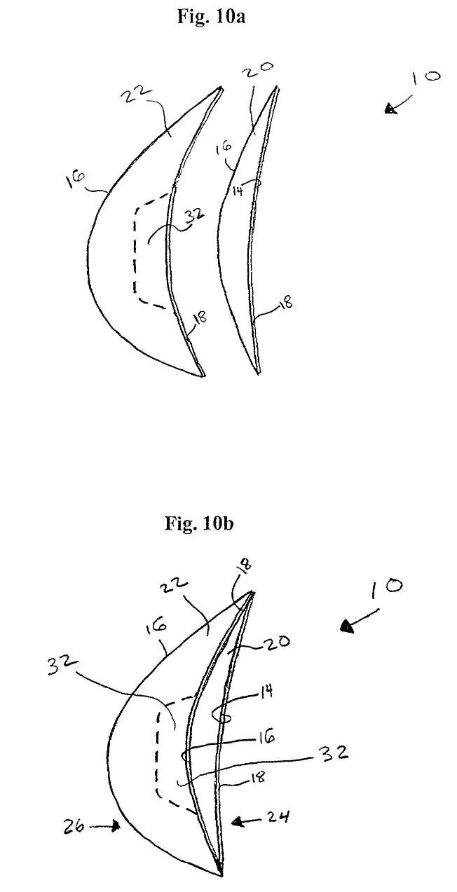

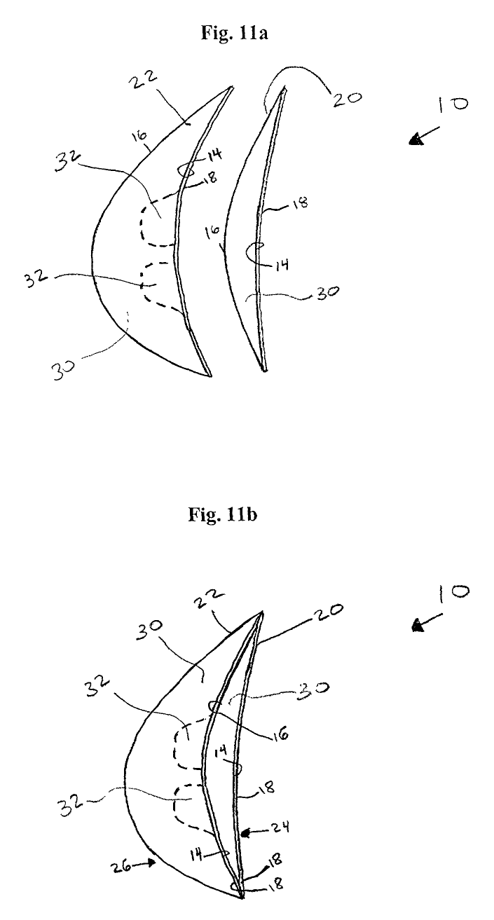

An additional feature of the breast form 10 is to provide one or more chambers 12 having void portions in the interior surface 14 of the chamber. Referring to FIGS. 10a-b, the front chamber 22 has a void portion 32 in the interior surface, which defines an indentation where the thickness between the exterior surface and the interior surface is reduced. Because the void portion 32 is an indentation, the interior surface 14 defines the void portion 32. It is optional to have the adhesive layer 18 permanently grown to the portion of the interior surface where the void portion 32 is defined. As shown in FIG. 10b, the interior surface where the void portion 32 is defined does not adjoin to the exterior surface 16 of the rear chamber 20. Therefore, the void portion 32 creates an air bubble between the front chamber and the rear chamber, which reduces the weight of the breast form. The void portion 32 preferably is not visible when the front chamber and rear chamber are adjoined as breast form 10. Accordingly, the void portion 32 provides a sort of invisible compartment, which is the user desired, could be used to store a personal belonging. As shown in FIGS. 11a-b, more than one void portion 32 can be created in any of the chambers. Although the void portion 32 is depicted only in the breast form 10 having two chambers, it is understood that any of the multiple chamber breast forms can make use of the feature of adding the void portion 32 to one or more chambers in order to reduce the weight of the breast form.

In addition to the specific features and embodiments described above, it is understood that the present invention includes all equivalents to the structures described herein, and is not to be limited to the disclosed embodiments. For example, the number of the chambers 12, and the sizing and configuration of the breast form 10 can be varied depending on the needs of the user. Individuals skilled in the art to which the present breast form pertains will understand that variations and modifications to the embodiments described can be used beneficially without departing from the scope of the invention.

What is claimed is:

1. A breast form comprising:
a plurality of independent and detachable chambers, each of the chambers formed from a thermoplastic film material;
a volume of deformable substance sealably disposed within each of the chambers; and
a pressure-sensitive adhesive permanently grown to an interior surface of each of the chambers, wherein the pressure-sensitive adhesive detachably adjoins to either an exterior surface of an adjacent chamber or a user's skin.

2. The breast form as recited in claim 1 wherein the plurality of chamber comprises a rear chamber positioned nearest the user's skin and a front chamber positioned farthest from the user's skin and adjacent the rear chamber.

3. The breast form as recited in claim 2 wherein the pressure-sensitive adhesive is permanently grown to the exterior surface of the rear chamber.

4. The breast form as recited in claim 2 wherein the deformable substance disposed within the front chamber is a silicone gel material.

5. The breast form as recited in claim 2 wherein the deformable substance disposed within the rear chamber is a foam.

6. The breast form as recited in claim 2 wherein the deformable substance disposed within the rear chamber is a lightweight silicone gel material comprising greater than about 40 percent by volume low-density filler.

7. The breast form as recited in claim 2 wherein the deformable substance disposed within the front chamber is a lightweight silicone gel material comprising greater than about 40 percent by volume low-density filler

8. The breast form as recited in claim 2 further comprising a middle chamber positioned between the rear chamber and the front chamber.

9. The breast form as recited in claim 8 wherein the deformable substance disposed within the middle chamber is air.

10. The breast form as recited in claim 8 wherein the deformable substance disposed within the rear chamber, the middle chamber, and the front chamber is different.

11. The breast form as recited in claim 1 wherein the pressure-sensitive adhesive has an adhesion force to the interior surface of the chamber to which it is permanently grown that is greater than a cohesion force to the exterior surface of the adjacent chamber or the user's skin to which it is detachably adjoined.

12. The breast form as recited in claim 1 wherein at least one of the plurality of chambers comprises a void portion in the interior surface, wherein adjoining the chamber having the void portion to the exterior surface of the adjacent chamber creates an air bubble between the interior surface of the chamber having the void portion and the exterior surface of the adjacent chamber.

13. A breast form comprising at least two independent and detachable chambers having a volume of substance sealed between an interior surface and an exterior surface opposite the interior surface, wherein the interior surface of each chamber has a layer of permanently grown pressure-sensitive, and at least a portion of the interior surface of one chamber detachably adjoins to at least a portion of the exterior surface of an adjacent chamber.

14. The breast form as recited in claim 13 wherein each of the chambers is formed of a thermoplastic film material.

15. The breast form as recited in claim 13 wherein the chambers comprises a rear chamber positioned nearest the user's skin and a front chamber positioned farthest from the user's skin and adjacent the rear chamber.

16. The breast form as recited in claim 15 wherein the layer of pressure-sensitive adhesive is permanently grown to the exterior surface of the rear chamber

17. The breast form as recited in claim 15 wherein the substance sealed within the front chamber is a silicone gel material.

18. The breast form as recited in claim 15 wherein the substance sealed within the rear chamber is a foam.

19. The breast form as recited in claim 15 wherein the substance sealed within the rear chamber is a lightweight silicone gel material comprising greater than about 40 percent by volume low-density filler.

20. The breast form as recited in claim 15 wherein the substance sealed within the front chamber is a lightweight silicone gel material comprising greater than about 40 percent by volume low-density filler.

21. The breast form as recited in claim 15 further comprising a middle chamber positioned between the rear chamber and the front chamber.

22. The breast form as recited in claim 21 wherein the substance sealed within the middle chamber is air.

23. The breast form as recited in claim 21 wherein the substance sealed within the rear chamber, the middle chamber, and the front chamber is different.

24. The breast form as recited in claim 13 wherein the layer of pressure-sensitive adhesive has an adhesion force to the interior surface of the chamber to which it is permanently grown that is greater than a cohesion force to the exterior surface of the adjacent chamber or the user's skin to which it is detachably adjoined.

25. The breast form as recited in claim 13 wherein at least one of the chambers comprises a void portion in the interior surface, wherein adjoining the chamber having the void portion to the exterior surface of the adjacent chamber creates an air bubble between the interior surface of the chamber having the void portion and the exterior surface of the adjacent chamber.

26. A method of using a breast form having a plurality of independent and detachable chambers, comprising the steps of:
adjoining a first chamber of the breast form to a user's skin, wherein an interior surface of the first chamber has a layer of permanently grown pressure-sensitive adhesive that removably attaches the first chamber to the user's skin; and
adjoining a second chamber to an exterior surface of the first chamber, wherein an interior surface of the second chamber has the layer of permanently grown pressure-sensitive adhesive that removably attaches the second chamber to the first chamber.
